(19) United States
(12) Patent Application Publication
(10) Pub. No.: US 2025/0082232 A1
(43) Pub. Date: Mar. 13, 2025

(54) DEVICE AND MOBILE BASED SYSTEM IN ORDER TO SCREENING AND DIAGNOSING THE LEVEL OF HEARING OF THE INFANTS AND CHILDREN BASED ON ANALYZING THE SIGNALS RECEIVED FROM BRAIN ON THE FUNCTIONAL NEAR-INFRARED SPECTROSCOPY (FNIRS) FOR DIAGNOSING THE POSSIBLE INFECTION OF THE MIDDLE EAR

(71) Applicant: Safa Samadzadeh Etehadi, Tehran (IR)

(72) Inventor: Safa Samadzadeh Etehadi, Tehran (IR)

(21) Appl. No.: 18/530,307

(22) Filed: Dec. 6, 2023

(51) Int. Cl. A61B 5/12; A61B 5/00; A61B 5/1171; G16H 50/20

(52) U.S. Cl. CPC A61B 5/125; A61B 5/002; A61B 5/0042; A61B 5/0075; A61B 5/1176; A61B 5/6803; A61B 5/7225; A61B 5/7275; A61B 5/746; G16H 50/20; A61B 2503/04; A61B 2562/0238

(57) ABSTRACT

The invention of device and mobile based system in order to screening and diagnosing the level of hearing of the infants and children based on analyzing the signals received from brain on the functional near-infrared spectroscopy (fNIRS) for diagnosing the possible infection of the middle ear includes at least one horseshoe headset. Generally, this invention relates to the structures and systems of measuring the hearing based on comparing the brain waves and also relates to systems of the monitoring and measuring the brain waves using the functional near-infrared spectroscopy technology and also relates to mobile based systems using artificial intelligence as an oscillator of generating sound waves and receiving feedbacks of the changes of the brain waves and analyzing the receiving signals to discover the possible infection of the hearing system in children.

### TECHNICAL FIELD OF THE INVENTION

The present invention relates to the structures and systems of measuring the hearing including structures and systems of measuring the hearing based on comparing the brain waves and also relates to systems of the monitoring and measuring the brain waves using the functional near-infrared spectroscopy technology and also relates to mobile based systems and methods of measuring the hearing using artificial intelligence as an oscillator of generating sound waves and receiving feedbacks of the changes of the brain waves and analyzing the receiving signals and also relates to a method to discover the possible infection of the hearing system in children.

### PRIOR ARTS OF THE INVENTION

The hearing process in human is the combination of the outer ear as the concentrator of the sounds, middle ear as the convertor of the kinetic energy of the sounds to the reciprocating motion of the set of auditory bones and inner ear as the convertor of the mechanical energy of the sound to electrical energy and performs to sending electrical signal to brain. In this mechanism, the middle ear is always at risk of being invaded by microorganisms due to the complexity of the movement mechanism of stapes, anvil and malleus bones and also due to being in a closed environment. Most of the times, the infection resulting from common diseases prevents the proper movement of the said bones and sometimes, caused formation of dense clotted tissue due to the excessive remaining of the infection in the middle ear. Generally, in adults the existence of infection in middle ear is diagnosed and treated quickly due to the correct understanding of the affected person about the decrease in hearing or the pain resulting from the presence of infection. However, this problem can cause extreme problems in children especially in infants, due to their inability to tell their pains or decrease of hearing. Among the mentioned problems, hearing loss or deafness of the child with the said problem can be mentioned. In this regard, audiometricians have to perform a tympanometry test to diagnose the problem of the affected person or the child and consider the reactions of the tested person as test results and analyze them. Since in audiometric tests with the help of a tympanometer, the tested person listens to the played sound through a headset or a device that creates vibrations in the lower bone of the skull and near the auditory canal, receives the vibrations created by sound waves with a certain frequency and on the other hand, can confirm hearing of the desired wave by pressing a button, therefore, the possibility of diagnosis is provided by this way in adults. However, given that this is not possible in infants and children and mostly the said child does not have the ability to determine and distinguish, the process of tympanometry testing or most audiometric tests is not possible in this range of children. In many cases, after the patient coming down, in case the infected person was a young child, the accumulation of infection in the middle ear and solidification of the infection in the middle ear can limit the mobility of the connected bones and further cause hearing loss in the infected child.

Many researchers have tried to create a method or an algorithm to detect the aforementioned infection before serious damage to the hearing system. In this regard, the use of electroencephalography or EEG technology has been used as one of the accurate diagnosis methods. An electroencephalogram (EEG) is a diagnostic test that measures the electrical activity of the brain using small metal discs called electrodes that are attached to the scalp. Brain cells communicate with each other through electrical impulses and are active any times, even during sleep. This activity is shown as wavy lines in the EEG graph. EEG is one of the main diagnostic tests for epilepsy and also it can be involved in diagnosing other brain disorders such as brain tumor, brain damage from head trauma, brain function disorders, sleep disorders, brain inflammation, stroke and Creutzfeldt-Jakob disease. It can also be used to confirm brain death in a person who is in a coma due to persistent brain problems. This test is safe and painless and you will feel little or no pain during it.

During the procedure, the small metal electrodes are attached to the scalp with thin wires. The electrodes detect tiny electrical charges that are caused by the activity of the brain cells. Usually the test takes between 20 and 40 minutes.

In this method, a specific number of electrodes are attached to the scalp on the skull with the help of a viscous gel with a high conductivity coefficient. Since external stimulants can generate signals in the auditory part and transmit them from the inner ear to the brain through the auditory nerve, it is logical that the electrical changes created in the brain or their effects can be observed in different parts of the brain, these electrical changes can be detected with the help of the EEG mechanism on the scalp and in a specific area. By creating different frequencies in the created sounds and sending these sounds to the person's ear, the brain signals are changed. In the EEG technique, the very small potential difference between the electrodes connected to the known channels with the stable level of electricity, which is the ground (harmful voltage), creates a specific wave form that depending on the number of electrodes placed on the patient's skull, the connected signals can be recorded on the output tape of the device or in computer files. By hearing the mentioned sounds by the test subject, regardless of age or speech abilities and common movements such as pressing a key or pointing, for each created signal, brain reflex and received EEG signal can be recorded. By examining the drawn graph using the EEG technique, the expert can identify the relationship between the produced sounds and the changes in the resulting graphs and further, by comparing the signals received with the help of EEG, determine the estimation of the hearing coefficient based on the sounds produced in each person and if there is an infection in the middle ear and through hearing loss in the affected person, the existing problem observe and solve before bigger problems arise. In this regard, various companies started customizing electroencephalography devices and integrating (combining) the EEG system with tympanometry equipment to achieve a kind of equipment and device to estimate the hearing level of a person and detect possible infections. Among the said items, we can mention a kind of headset produced by Emotiv Company under the title of fourteen-channel EEG device. Certainly, the mentioned device is only a type of portable electroencephalography, which is not claimed to be able to be combined with a tympanometry device, but it is obvious that both devices can be used simultaneously if needed. In another device provided by the same company, a five-channel wireless headset called insight has been presented, which has the ability to receive waves from two electrodes on the back of the head, two electrodes on the sides of the forehead, and one electrode on the upper part of the head and it can collect and connect to any brain wave stimulation system and receive the resulting electric wave reflexes if needed.

In this regard, it can mention to the below inventions, some of which are combination of two or more techniques to stimulate and analyze the amount of excitability of the brain due to receiving said waves:

In an US invention with patent number U.S. Pat. No. 6,154,669 which is granted dated 28 Nov. 2000 titled “Headset for EEG measurements” provides headset and system for taking EEG measurements on the head of a test subject. The headset has a plurality of electrode sensors in predetermined anatomical positions on the head of a subject to take EEG measurements, for example of the subject's response to certain stimuli. The electrode sensors are formed of dry conductive foam rubber pads attached to conductor elements. An individual shielded cable is attached to each sensor to carry the detected signal to a signal processor. The sensors may be formed of a carbon impregnated foam rubber pad that offers high conductivity. The sensors do not require wetting agent, and thus can be used in a very time efficient manner, and can be easily replaced for sanitary considerations.

A Chinese invention with patent number CN2824836Y which is granted dated Oct. 11, 2006 titled “Head-mounted physiological parameter measuring device” relates to a head mounted physiological parameter measuring device, particularly a head mounted device used for realizing the measurement of a plurality of physiological parameters. The utility model comprises a head mounted device, at least one sensor, a processing unit, a receiving unit and a notifying unit, wherein the head mounted device is fixed to the ears or the head of a person who wears the head mounted device; the sensor(s) is (are) positioned at a proper position of the head mounted device, so that when the head mounted device is used, the sensor(s) is (are) close to the pinnas or the external auditory meatus of the person who wears the head mounted device to sense physiological signals and convert the physiological signals into electrical signals; the processing unit receives the electrical signals from the sensor(s) and calculates a plurality of physiological parameters according to the electrical signals; the receiving unit receives data inputted by the person who wears the head mounted device to assist the processing unit in calculating the physiological parameters; the notifying unit notifies the person who wears the head mounted device the measured physiological parameters in a displaying mode or in a voice prompt mode. The utility model realizes the measurement of a plurality of key physiological parameters, detected physiological parameters can be transmitted to the notifying unit in a wireless transmission mode and measuring switches can be controlled by biological signals.

Another Spanish invention with patent number ES2320942T3 which is granted dated 29 May 2009 titled “EAR EXAM DEVICE WITH TEMPERATURE SENSOR” is a medical instrument for analyzing the ear of a subject, comprising: a temperature sensor to measure the subject's body temperature and provide a first signal indicative thereof; a fluid detector to detect the presence of fluid in the ear to provide a second signal indicative thereof; and a controller connected to the temperature sensor and the fluid detector to control the acquisition of the first and second signals.

In a Japanese invention with publication number JP2022002157A which is filed dated 15 Oct. 2021 titled “Device, method and system for obtaining medical diagnostic information, and provision of remote medical service” provide a device, a method, a system, and a remote medical service, for obtaining medical diagnostic information. The present invention relates to various systems, tools and methods, which are provided for users for: obtaining diagnostic information including medical information; transmitting the information to remote areas; evaluating the information; and transmitting consequent diagnostic and therapeutic information to the users and/or third parties for following actions. The present invention provides remote therapeutic systems and procedures useful for consumers and the users, which attain to provide health management services and/or diagnosis remotely from afar.

In another US invention with patent number U.S. Pat. No. 6,556,852 which is granted dated 29 Apr. 2003 titled “Earpiece with sensors to measure/monitor multiple physiological variables” provides an apparatus and method for positioning sensors relative to one another and anatomic features in a non-invasive device for measuring and monitoring multiple physiological variables from a single site uses an earpiece incorporating a shielded pulse oximetry sensor (POS) having a miniaturized set of LEDs and photosensors configured for pulse oximetry measurements in the reflectance mode and located in the earpiece so as to position the POS against a rear wall of an ear canal. The earpiece also includes a thermopile of no larger than 7 mm. in diameter located on the earpiece to so as to position the thermopile past a second turn of an external auditory meatus so as to view the tympanic membrane. The thermopile includes a reference temperature sensor attached to its base for ambient temperature compensation.

Another US invention with patent number U.S. Pat. No. 8,702,607 which is granted dated 22 Apr. 2014 titled “Targeted advertising systems and methods” systems and methods for monitoring various physiological and environmental factors, as well as systems and methods for using this information for a plurality of useful purposes, are provided. Real-time, noninvasive health and environmental monitors include a plurality of compact sensors integrated within small, low-profile devices. Physiological and environmental data is collected and wirelessly transmitted into a wireless network, where the data is stored and/or processed. This information is then used to support a variety of useful methods, such as clinical trials, marketing studies, biofeedback, entertainment, and others.

In another US invention with patent number U.S. Pat. No. 8,858,430 which is granted dated 14 Oct. 2014 titled “Ear ailment diagnostic device and method” is an ear ailment diagnostic device and method that generally comprises a pair of earpieces, which both further comprise a light source, a magnification lens, an air conduction channel and a miniature camera. The earpieces may optionally comprise a thermometer and/or tympanometer. Each earpiece is coupled to an air conduction tube, an insufflator and an electrical wiring/data tube which is coupled to a computer. The insufflator may be manually, electronically, or battery powered. In the preferred embodiment the computer comprises a smart phone with data processing capability and wireless communication capability. Any data sent from the device can then be interpreted and diagnosed in a remote location so that an accurate treatment is prescribed.

An US invention with patent number U.S. Pat. No. 9,491,277 which is granted dated Aug. 11, 2016 titled “Computerized method and system for global health, personal safety and emergency response” is a headband and computerized system and method for intuitively detecting a user's need for emergency help, preventive care and medical interventions and also for coordinating care for user's loved ones and pets when user experiences an emergency or has passed away. Detection is done via labs and life signs equipment and sensors, self-initiated request for monitoring or the system's periodically polling user through an alert, a telephone call, a notification, a text message or other appropriate means to find out if they are in danger. Polling and automated monitoring may also be triggered by real-time medical data electronically or wirelessly transmitted to the system. If user fails to respond, the system automatedly calls either user's contacts (by listed priority) to notify them of a potential emergency, or emergency responders directly depending on severity of the emergency and communicates to them critical information as well as a code to access user's medical record(s). The system may also receive real-time data on the user's medical condition via medical monitoring devices, such as the user's vital signs (heart rate, respiration, blood pressure, temperature, oxygen saturation), EKG, blood glucose, other blood work and tests such as blood sugar, and other labs such as drug level, anemia, urinalysis, bacteria in urine, bacteria in the blood, strep throat, ear infections.

In an US invention with patent number U.S. Pat. No. 10,575,777 which is granted dated Mar. 3, 2020 titled “In-ear electrical potential sensor” provide an audio product for obtaining biologically-relevant information associated with a user comprising. The audio product includes at least two electrodes, a processor, and an electroacoustic transducer coupled to the processor. The processor is configured to receive at least one signal affected by an action of the user obtained via the first electrode or the second electrode and take one or more actions based on the at least one signal. The at least one action may control another device, in an effort to provide hands-free control of the other device. According to an aspect, the at least one signal comprises an electromyogram (EMG), electrooculogram (EOG), or electroencephalogram (EEG) associated with the user.

Another US invention with patent number U.S. Pat. No. 11,045,092 which is granted dated 29 Jun. 2021 titled “Apparatus and method for measuring biologic parameters” is a support structures for positioning sensors on a physiologic tunnel for measuring physical, chemical and biological parameters of the body and to produce an action according to the measured value of the parameters. The support structure includes a sensor fitted on the support structures using a special geometry for acquiring continuous and undisturbed data on the physiology of the body. Signals are transmitted to a remote station by wireless transmission such as by electromagnetic waves, radio waves, infrared, sound, and the like or by being reported locally by audio or visual transmission. The physical and chemical parameters include brain function, metabolic function, hydrodynamic function, hydration status, levels of chemical compounds in the blood, and the like. The support structure includes patches, clips, eyeglasses, head mounted gear and the like, containing passive or active sensors positioned at the end of the tunnel with sensing systems positioned on and accessing a physiologic tunnel.

And another US invention with patent number U.S. Pat. No. 6,773,405B2 which is granted dated Oct. 8, 2004 titled “Ear temperature monitor and method of temperature measurement” is a continuous body core temperature monitor comprises a pliable ear plug that conforms to the shape of an ear canal and incorporates a temperature sensor that is clamped between the plug and the ear canal wall. The external surface of the plug is connected to an external temperature sensor and a heating element that compensate for a heat lost from the ear canal to the environment by maintaining the temperature gradient between the temperature sensor and the heating element close to zero. The external temperature sensor is for measuring temperature of said enclosure; thermal insulator positioned between temperature transducer and external temperature sensor and electronic module connected to said temperature transducer and external temperature sensor.

An US invention with publication number US20020188216A1 which is filed dated Mar. 5, 2002 titled “Head mounted medical device” is a head mounted medical device for obtaining and processing EEG, EKG, and EOG/EMG signals from a wearer. The device utilizes electrodes organized into multiple electrode assemblies for ease of use and replacement, with the electrode assemblies being removably connected to the headband. The device includes processing and conditioning circuitry in the headband, the processing and conditioning including amplification, filtering, A/D conversion, and multiplexing of the analog signals generated by the electrodes, reducing noise influences and improving the handling and mobility of the device. The device connects to an external receiving device that then monitors and/or records the resulting serial, digital, and multiplexed data signal. The external device might also provide power and/or commands to the medical device. In this invention each electrode signal is an analog electrode signal, and further wherein said headband circuit converts each analog electrode signal into a digital format all being multiplexed together into said EEG data signal output by said device, said EEG data signal being a serial data signal.

An US invention with publication number US20030220584A1 which is filed dated 18 Apr. 2003 titled “Headset for measuring physiological parameters” is a methods and systems for determining physiological parameters from body sounds obtained from a person's ear. In various exemplary embodiment, the system includes an earplug housing; a sensing element disposed within a portion of the earplug housing; an acoustic shield coupled to the earplug housing, the acoustic shield reducing or eliminating extracorporeal sounds; and a preamplification circuit electrically coupled to the sensing element. In various exemplary embodiments, the system is operable to determine motion and/or vibration of the external acoustic meatus or the tympanic membrane of the ear due to internally generated body sounds.

Another US invention with patent number U.S. Pat. No. 8,565,852B2 which is granted dated 22 Oct. 2013 titled “Auricle-installed device and bio-signal measurement apparatus” is an auricle-installed device including, a reference electrode to be placed on the ear lobe, a detection electrode to be placed on a surface exposed to bones surrounding the auricle of the ear, and a support body for supporting the reference and detection electrodes. said support body comprises a main section formed to fit the base of said auricle and provided with said reference electrode on one end of said main section; and a flexible wire-shaped member for connecting said main section to said detection electrode; and said detection electrode has a structure that can be held in a hollow between the crus of helix of said auricle and the superior crux of antihelix of said auricle. A bio-signal measurement apparatus comprising an auricle-installed device employing a reference electrode to be placed on the ear lobe, a detection electrode to be placed on a surface exposed to bones surrounding the auricle of said ear as well as a support body for supporting said reference and detection electrodes; and an amplifier for amplifying a difference in electric potential between said reference and detection electrodes as a bio-signal.

And also in an US invention with publication number US20110004089A1 which is filed dated May 2, 2009 titled “Ear-worn EEG monitoring device” an ear-worn EEG monitoring device is disclosed. The EEG monitoring device includes plural electrodes, an EEG signal acquisition circuitry with RF module, at least a housing for accommodating the EEG signal acquisition circuitry with RF module, and at least an ear-worn structure, wherein the ear-worn structure can be implemented to mount on one or two ear(s) of the user, so that during the EEG monitoring process, the ear-worn structure can be used to hold and support the housing and the EEG signal acquisition circuitry therein at position(s) above and including the neck of the user, and the RF module is used for achieving a wireless communication with an external apparatus, so as to transmit acquired signals thereto.

Functional Near-Infrared Spectroscopy (fNIRS) is a non-invasive brain monitoring technique that uses near-infrared light to assess cortical hemodynamic activity. This technique measures oxygen and deoxygen concentration changes in hemoglobin, blood volume and oxygenation, and oxygen metabolic rate. fNIRS is one of the most common non-invasive photo imaging techniques that can be used in portable fields. This technique is safe, non-invasive and can be used with other imaging methods. fNIRS is a non-invasive imaging technique that involves measuring the concentration of dissolved chromophores by measuring near-infrared light reduction or time or phase changes. This technique uses an optical window where skin, tissue, and bone are most transparent to near-infrared light (700-900 nm spectral range) and hemoglobin (Hb) and deoxygen hemoglobin (deoxy-Hb) are strong light absorbers. fNIRS is capable of measuring changes of concentrations in both hemoglobin oxygen and hemoglobin deoxygen, but can only measure from regions near to the cortical surface. In common technologies for detecting or measuring physical or electrical changes in common body tissues, fNIRS technology can be mentioned. In this technology, generally, the existence of a source of generating visible light waves or invisible light with a specific frequency is used as the primary wave transmitter. The light waves have the capability to penetrate to different body tissues in case of having enough wave intensity as well as high frequency. By entering the light waves to the tissue, the reflexion of these waves can exit from tissues and the energy of the light packs converts to the electrical pulse through intensity assessment and output light frequency sensor. Here, the magnification of the form of the output waves by amplifying the output signals can be possible. If the intensity of magnification is on a rational scale, it is possible to detect the resulting changes and refer the created frequency to a wave detector.

### DESCRIPTION OF THE INVENTION

The present invention is a kind of hearing assessment and diagnosis system in children to diagnose infectious diseases of the inner ear or auditory nerve passages and channels. In this invention, the headset is independent from the network energy flow, such as city electricity flow, which has an energy storage source, such as lithium batteries (FIG. 1, No. 105) can be charged when not in use and ready for use when needed. The mentioned headset has the horseshoe shape that is designed in such a way that it has a flexible polymer structure in the four areas of the front side curves (FIG. 1, No. 108) and also at the end of the back part of the head (FIG. 1, No. 112). So, at the time of placing the horseshoe shape headset on the child's skull, the presence of four flexible points equipped with blade springs caused the horseshoe shape of the headset stick to the skull and finally, the five rear parts, the rear sides and the front sides should be attached to the skull and placed at the smallest distance. In most headsets, in order to control weight of the device, the headset has to be placed from the top of the head down, however in this invention due to the need to use and communicate with the electrical channels of the rear part of the brain and to make the device smaller and lighter, the horseshoe shape is placed around the skull and in the rear part of the head, and the device has a movable upper attachment (FIG. 1, No. 101) which is placed in its mounted section in the headset shell when not in use and during use, by moving around its joints, it is rotated by 90 degrees and placed perpendicular to the headset, and as a holding attachment it can cause the device to be fixed on the child's head and not slipping and moving down the headset. The said attachment has an ergonomic shape and suitable for children's form of skull. In the headset of the present invention the fNIRS technology is used to receive the changes of shape and brain waves frequency. The said device has three separate parts for sending and receiving light beam and receiving the required feedback. In the front part, at the head of each of the palpus, there is a light source to produce infrared light (FIG. 1, No. 104 and 107) with a frequency of (700-900 nm) which is equipped with a receiver (FIG. 1, numbers 103 and 106) near and next to it. Also, in the rear part of the headset, a set of transmitter (FIG. 1 No. 113) and receiver (FIG. 1 No. 114) is placed right in the middle and behind the skull. During the operation of the headset, infrared light is produced from the transmitter of all three measurement channels and sent to the bone tissue of the skull. Normally, infrared light with frequency of (700-900 nm) can penetrate into the skull tissue and reflect without any harmful effects or causing problems after hitting the tissues of the scalp, skull bone and soft tissue of the brain and the integration of the reflection is received by the receiver which is next to the transmitter. Changes in brain waves that are carried out due to the communication of different parts of the brain with each other and shows itself as wave changes in electroencephalography, in fact is an index to measure the changes in the return of the transmitted infrared light beam. In this device, by sending sound waves from a source connected to the device, which can be a smart mobile phone (FIG. 5), the required stimulation is done on the inner ear and sending sounds to the brain. After sending sound waves to the child's ear, changes in the electrical frequencies of the hearing part of the brain cause changes in the output of infrared light waves sent to the brain that these changes are received by the receiving sensor of each of the headset channels separately and are led as an electrical signal to the electrical board section (FIG. 1, No. 109) of the headset. In the headset part of the present invention, the received waves are amplified with the help of the amplifier circuit in the pcb of the device and in the form of sendable data sent from the headset to the mobile phone using the Bluetooth protocol. Whole of the mentioned process, including sending a specific sound wave with a specific frequency and strength to the child's ear, controlling the headset, receiving the output signals from the headset, and preparing the required information packs are carried out on the smartphone application. The smart mobile phone is connected to an artificial intelligence server from the beginning of the process, where all executable programs are located on this server, and the management of the process is the responsibility of AI. All hearing assessment protocols in the structure of tympanometry are designed, executed and feedbacked by artificial intelligence, artificial intelligence may repeat part of the test depending on the results obtained and summarize the results as a criterion, during the hearing test of the child's ears. Standard tests are dictated and executed separately by AI to the device, and the feedback of brain wave changes is sent to the AI server. Now, by comparing different frequency bands in the range of human hearing and depending on the age, gender and previous records of the assessment made by the device, AI can make a meaningful comparison between the changes made or the results obtained. By using the face recognition mechanism in the mobile phone at the beginning of using the device for a child, the child's face photo can be taken and in every enter, by scanning the child's face, the related file will be called and all steps will be done in their account. This important issue helps to use the device in families with several children or in a more professional manner in children's clinics. At the end of the hearing test and receiving the map of one of the reflexes of the child's brain system and comparing the information obtained from the changes in the brain waves, the AI will be able to draw the required graphs for the changes of each channel of the device and further, with the help of available database in AI, it can determine the child's hearing level in each of the frequency bands. Now, if there is an infection in the inner ear and a significant decrease in hearing in some hearing areas, the artificial intelligence system has monitored the mentioned changes and after the required analysis, if the child is in the high-risk range in terms of the presence of an infection in the inner ear, the required information is first sent as an alert to the smart mobile phone on the existing communication platform and it can also send the possibility of the risk of infection in the inner ear to the family doctor and pediatrician. With the start of the notification protocol, if hearing loss is diagnosed due to the onset of an inner ear infection, the process of referring the child to a specialist doctor and receiving the correct dose of antibiotics, if necessary, begins. During the treatment period, repeating the assessment process can indicate the progress and improvement of the treatment.

### BRIEF DESCRIPTION OF FIGURES

FIG. 1 shows the overall 3D view of the headset.

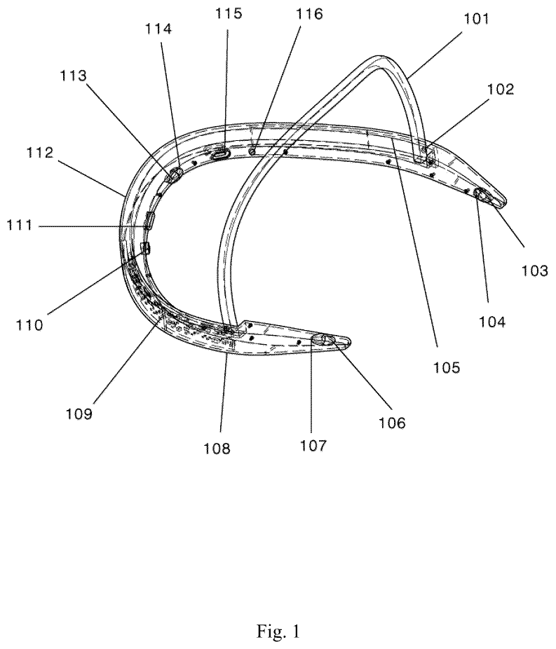

FIG. 2 shows the exploded view of the headset.

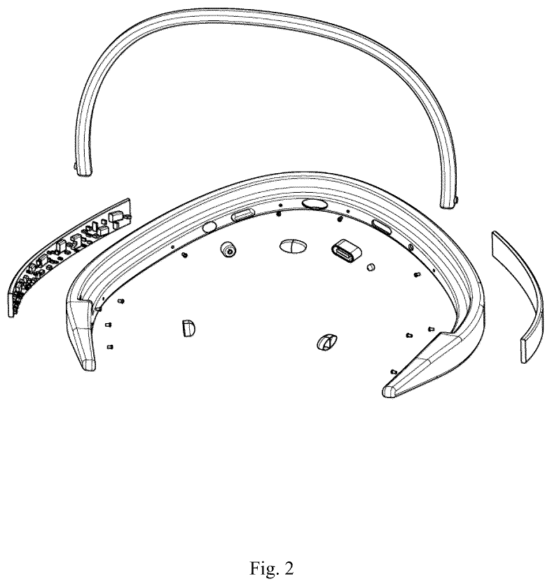

FIG. 3 is a 3D view of the placement of the headset on the child's head.

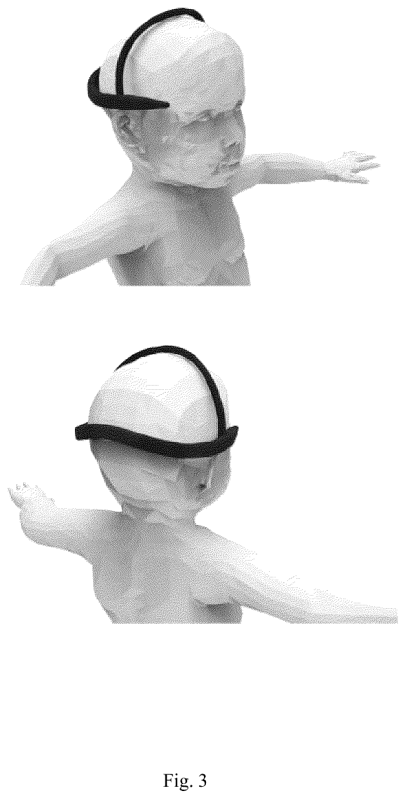

FIG. 4 shows the front, left, top and three-dimensional views of the headset.

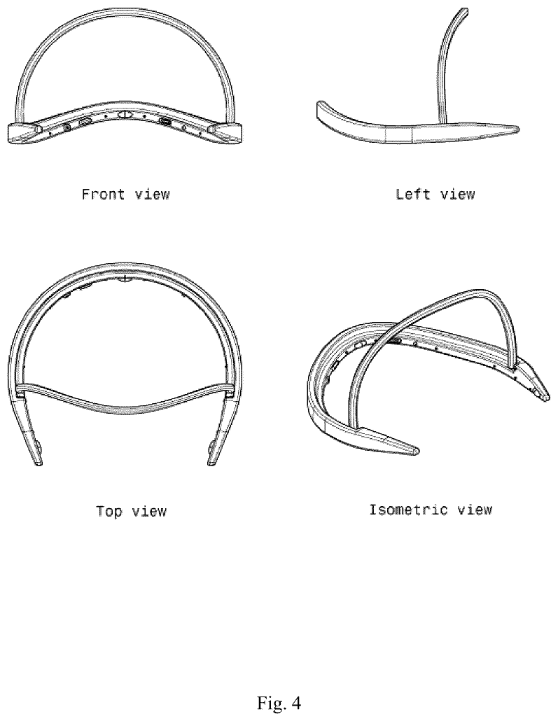

FIG. 5 shows connecting the headset to the smart phone and then connecting to the artificial intelligence server.

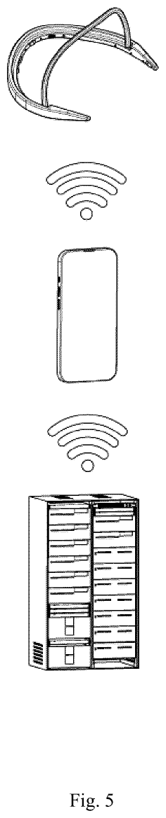

FIG. 6 shows the 3D view of the infrared sensors and the receiver.

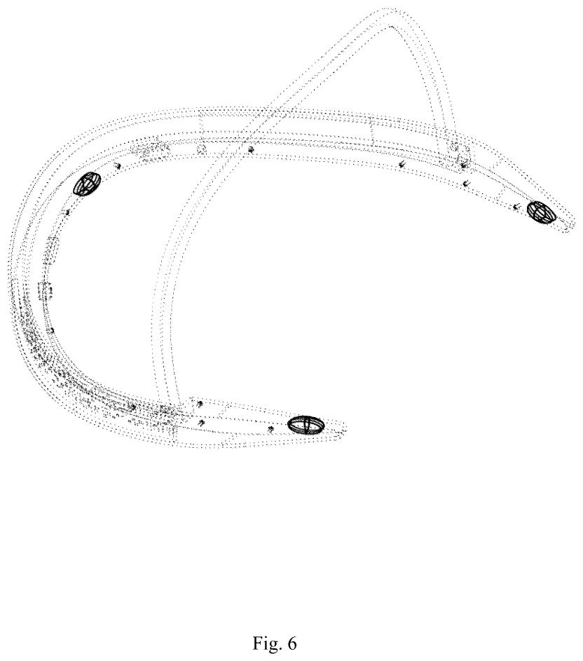

FIG. 7 shows the 3D view of the headset battery.

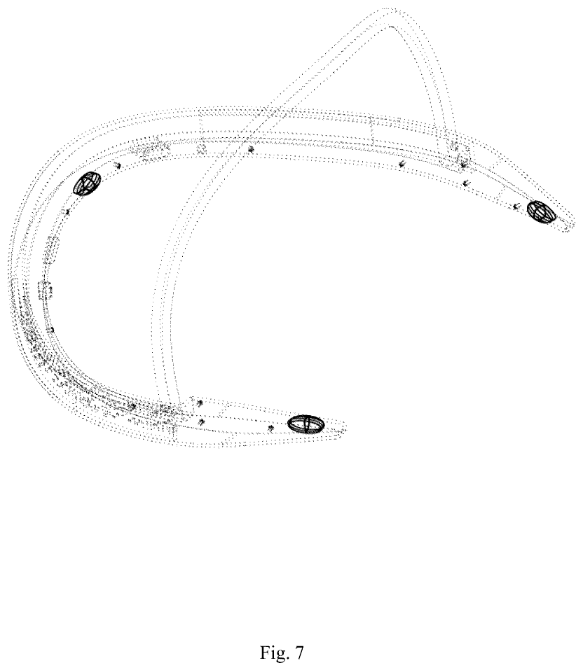

FIG. 8 shows the 3D view of the headset board.

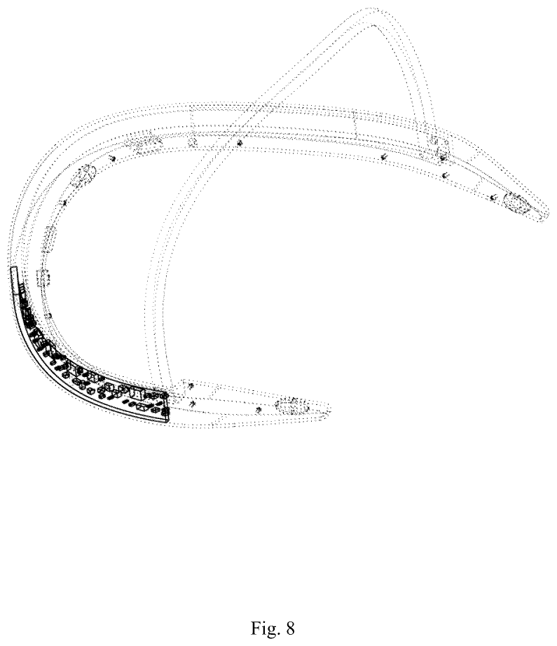

What is claimed is:

1. The invention of device and mobile based system in order to screening and diagnosing the level of hearing of the infants and children based on analyzing the signals received from brain on the functional near-infrared spectroscopy (fNIRS) for diagnosing the possible infection of the middle ear includes at least one horseshoe headset and at least one lithium battery that the headset is connected to and at least four curves with a polymer structure and at least one movable upper attachment and at least one holding attachment and at least one or more palpuses and at least one light source to produce infrared light and at least one set of receivers and transmitters and at least one source of sending sound waves and at least one electric board and at least one amplifier circuit and at least one Bluetooth protocol and at least one smartphone application and at least one artificial intelligence server.

2. The device and mobile based system of claim 1 in which the headset has the horseshoe shape that is designed in such a way that it has a flexible polymer structure in the four areas of the front side curves and also at the end of the back part of the head.

3. The device and mobile based system of claim 1 in which four flexible points are equipped with blade springs and caused the horseshoe shape of the headset stick to the skull.

4. The device and mobile based system of claim 1 in which due to use and communicate with the electrical channels of the rear part of the brain, horseshoe shape is placed around the skull and in the rear part of the head.

5. The device and mobile based system of claim 1 in which the device has a movable upper attachment which is placed in its mounted section in the headset shell when not in use and during use, by moving around its joints, it is rotated by 90 degrees and placed perpendicular to the headset, and as a holding attachment it can cause the device to be fixed on the child's head and not slipping and moving down the headset.

6. The device and mobile based system of claim 1 in which the device has three separate parts for sending and receiving light beam and receiving the required feedback.

7. The device and mobile based system of claim 1 in which in the front part, at the head of each of the palpus, there is a light source to produce infrared light with a frequency of (700-900 nm) which is equipped with a receiver near and next to it.

8. The device and mobile based system of claim 1 in which in the rear part of the headset, a set of transmitter and receiver is placed right in the middle and behind the skull.

9. The device and mobile based system of claim 1 in which during the operation of the headset, infrared light is produced from the transmitter of all three measurement channels and sent to the bone tissue of the skull.

10. The device and mobile based system of claim 1 in which by sending sound waves from a source connected to the device, which can be a smart mobile phone, the required stimulation is done on the inner ear and sending sounds to the brain.

11. The device and mobile based system of claim 1 in which after sending sound waves to the child's ear, changes in the electrical waves of the hearing part of the brain cause changes in the output of infrared light waves sent to the brain that these changes are received by the receiving sensor of each of the headset channels separately and are led as an electrical signal to the electrical board section of the headset.

12. The device and mobile based system of claim 1 in which the received waves are amplified with the help of the amplifier circuit in the pcb of the device and in the form of sendable data sent from the headset to the mobile phone using the Bluetooth protocol.

13. The device and mobile based system of claim 1 in which whole the process including sending a specific sound wave with a specific frequency and strength to the child's ear, controlling the headset, receiving the output signals from the headset, and preparing the required information packs are carried out on the smartphone application.

14. The device and mobile based system of claim 1 in which the smart mobile phone is connected to an artificial intelligence server from the beginning of the process, where all executable programs are located on this server, and the management of the process is the responsibility of AI.

15. The device and mobile based system of claim 1 in which all hearing assessment protocols in the structure of tympanometry are designed, executed and feedbacked by artificial intelligence.

16. The device and mobile based system of claim 1 in which standard tests are dictated and executed separately by AI to the device, and the feedback of brain wave changes is sent to the AI server.

17. The device and mobile based system of claim 1 in which by using the face recognition mechanism in the mobile phone at the beginning of using the device for a child, the child's face photo can be taken and in every enter, by scanning the child's face, the related file will be called and all steps will be done in their account.

18. The device and mobile based system of claim 1 in which at the end of the hearing test and receiving the map of one of the reflexes of the child's brain system and comparing the information obtained from the changes in the brain waves, the AI will be able to draw the required graphs for the changes of each channel of the device and further, with the help of available database in AI, it can determine the child's hearing level in each of the frequency bands.

19. The device and mobile based system of claim 1 in which after the required analysis, if the child is in the high-risk range in terms of the presence of an infection in the inner ear, the required information is sent as an alert to the smart mobile phone or to the family doctor and pediatrician on the existing communication platform.
